(19) United States
(12) Patent Application Publication
(10) Pub. No.: US 2021/0068788 A1
(43) Pub. Date: Mar. 11, 2021

(54) METHODS AND SYSTEMS FOR A MEDICAL IMAGING DEVICE

(71) Applicant: GE Precision Healthcare LLC, Milwaukee, WI (US)

(72) Inventors: Jiajiu Yang, Wuxi City (CN); Dongwei Wang, Wuxi City (CN); Rong Li, Wuxi City (CN); Yi Wang, Wuxi City (CN); Weiwei Zhou, Wuxi City (CN)

(21) Appl. No.: 16/566,807

(22) Filed: Sep. 10, 2019

(51) Int. Cl. A61B 8/08; A61B 8/00

(52) U.S. Cl. CPC A61B 8/483; A61B 8/4254; A61B 8/085; A61B 8/5223; G06T 2207/10132; A61B 8/5207

(57) ABSTRACT

Various systems are provided for an ultrasound imaging device. In one example, a method comprises instructing a user a location to which to navigate an ultrasound probe in one or more of a horizontal plane and a vertical plane based on a previously acquired first image at a first anatomical location while acquiring a second image at a second, different anatomical location and automatically joining the first and second images together.

### TECHNICAL FIELD

Embodiments of the subject matter disclosed herein relate to medical imaging and the facilitation of ultrasonic tissue scanning.

### DISCUSSION OF ART

In ultrasonic diagnostic apparatuses, ultrasound is transmitted from an ultrasonic probe to the inside of a subject, and echo signals reflected from the inside of the subject are received by the ultrasonic probe. Then, an ultrasonic image is produce based on the received echo signals and displayed on a display device.

In some ultrasonic diagnostic apparatuses, transmission/reception of the ultrasound is performed on a 3D region to acquire ultrasonic volume data. The volume data is in some cases acquired by a 3D probe where an operator changes an angle or a pressure of the ultrasonic probe or moves the ultrasonic probe to perform transmission/reception of an ultrasound on a 3D region and acquire ultrasonic volume data.

### BRIEF DESCRIPTION

In one embodiment, a method, comprises instructing a user to navigate an ultrasound probe via a movement in a horizontal plane or a pressure in a vertical plane based on a previously acquired first image at a first anatomical location while acquiring a second image at a second, different anatomical location and automatically joining the first and second images together.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows an example of an ultrasound imaging system according to an embodiment of the disclosure;

FIG. 2 shows an example schematic of various system components of an ultrasound imaging system according to an embodiment of the disclosure.

FIG. 3 shows a high-level flow chart for joining two ultrasound images.

FIG. 4 shows a method for acquiring a first ultrasound image and a second ultrasound image.

FIG. 5 shows an example of an ultrasound image acquisition to join with a previously acquired ultrasound image.

FIG. 6 shows an example of a previously acquired ultrasound image being joined with a current ultrasound image.

FIG. 7 shows a displacement of an ultrasound probe along a horizontal plane.

### DETAILED DESCRIPTION

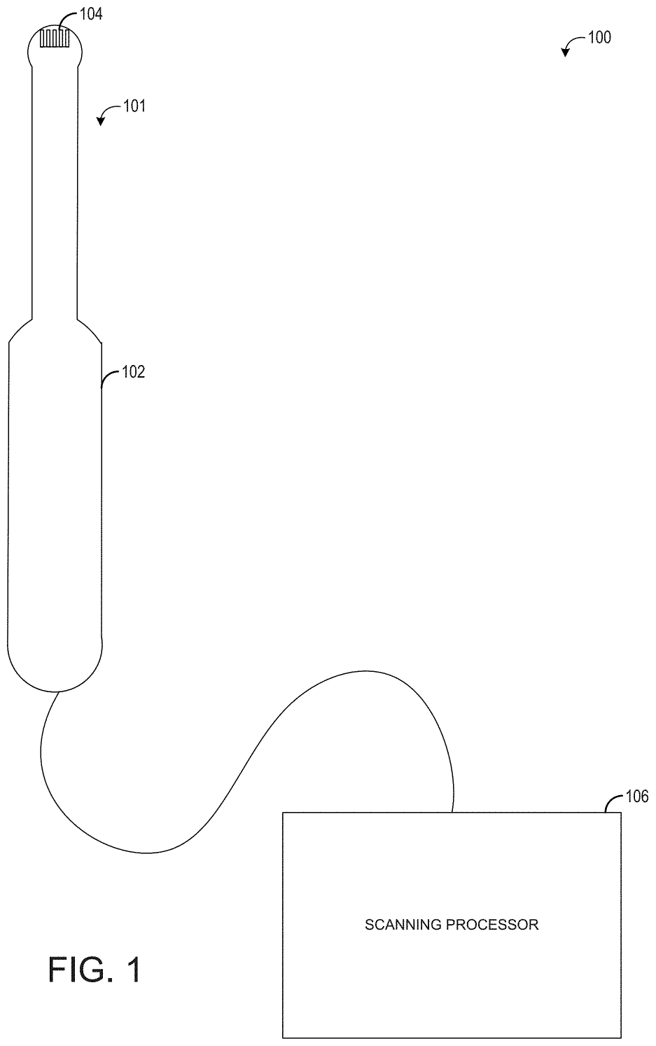

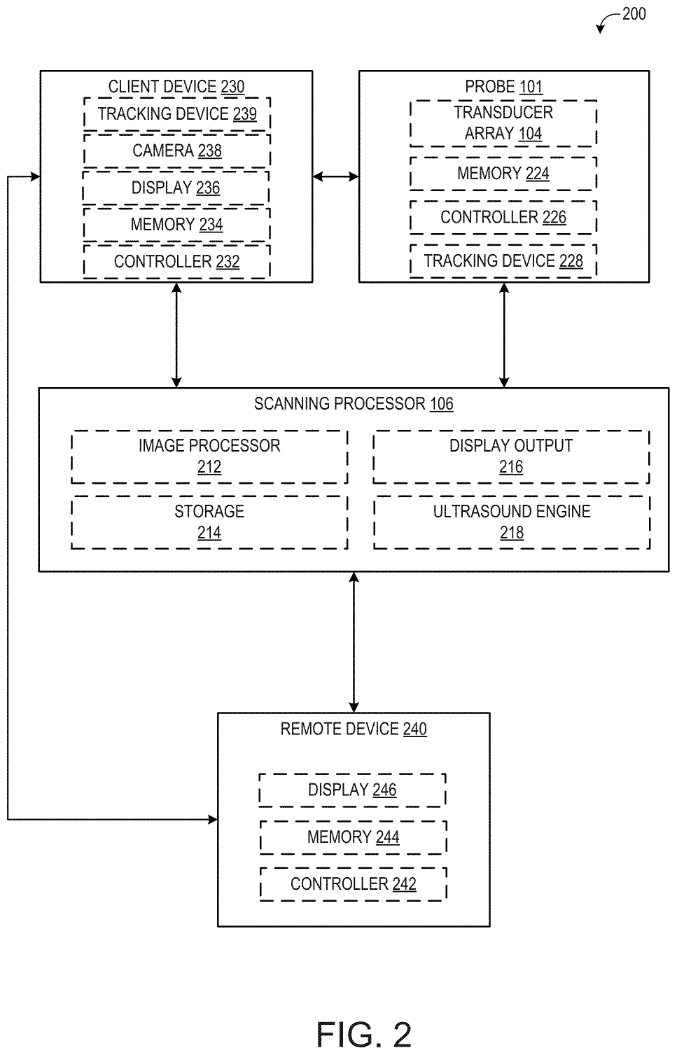

The following description relates to embodiments of an ultrasound device configured to combine two or more ultrasound images. An example of the ultrasound image system is shown in FIG. 1 and schematics of various system components of the ultrasound system are shown in FIG. 2. The ultrasound system of FIG. 2 may be configured to acquire 3D ultrasound images. In one example, 3D ultrasound images are generated via a plurality of slices of a volume data. When scanning larger areas, it can be difficult for the user to visualize how to position the ultrasound probe in order to generate adjacent images that can be stitched together because of the nature of the volume data and image generation from the volume data. As such, in some examples, a user interface may provide instructions to the user as to how to move, position, and apply pressure with, the ultrasound probe so as to improve the ability to generate multiple images from the overall volumetric data that can be stitched together to form a larger image from the volume data acquired over time as the probe moves.

Each slice of the image may correspond to a time and a spatial dimension in which an anatomical area is scanned to generate volume data. The ultrasound system may analyze slices acquired or formed from different sets of volume data, wherein reference points are marked on each slice. Slices with matching and/or overlapping reference points may be stitched together to form a larger, whole image relative to the plurality of slices segmented from one another.

The ultrasound device may be configured to identify reference points in two or more diagnostic images generated from volume data based on a deep learning neural network and/or an algorithmic method. The diagnostic images may be generated from different sets of volumetric data acquired at different times and positions and pressure levels. For example, from two sets of volume data, more than two images may be generated from which two images can be selected whose reference points meet selected criteria. The reference points may be used to identify reference pairs between the two or more images. In this way, if at least two images comprise a reference pair, then the two images may be merged together along respective edges, such that the images do not overlap but touch along the edges to form one final, continuous image.

By merging volumetric data acquired at different times and positions and pressure levels via stitching 2-D images, quantitative measures, such as chamber volumes, accumulation of fluid, and dynamic movement of blood cells may be better viewed by the user. Additionally or alternatively, the large image may be rotated or cropped in real-time to reveal anatomical structures within a volume of tissues, which may be beneficial in the treatment of cancers and the like. Further still, but instructing the user how to position, apply, and/or maneuver the probe, improvements in the combined larger 2-D images may be provided.

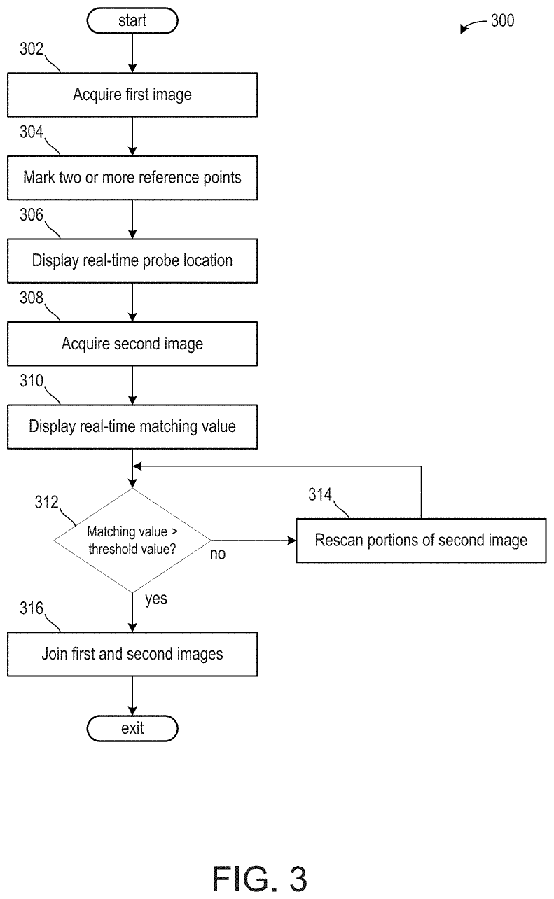

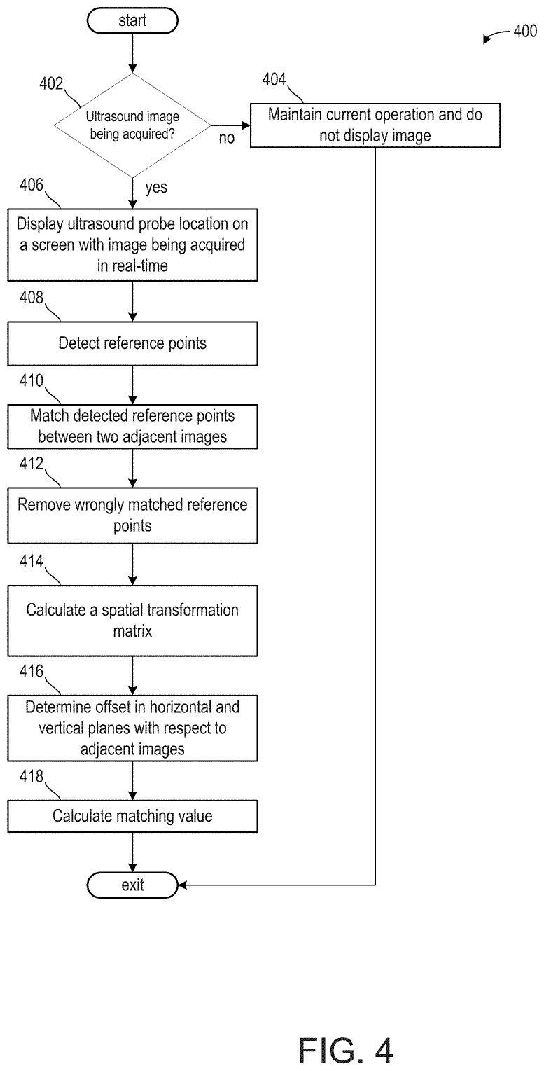

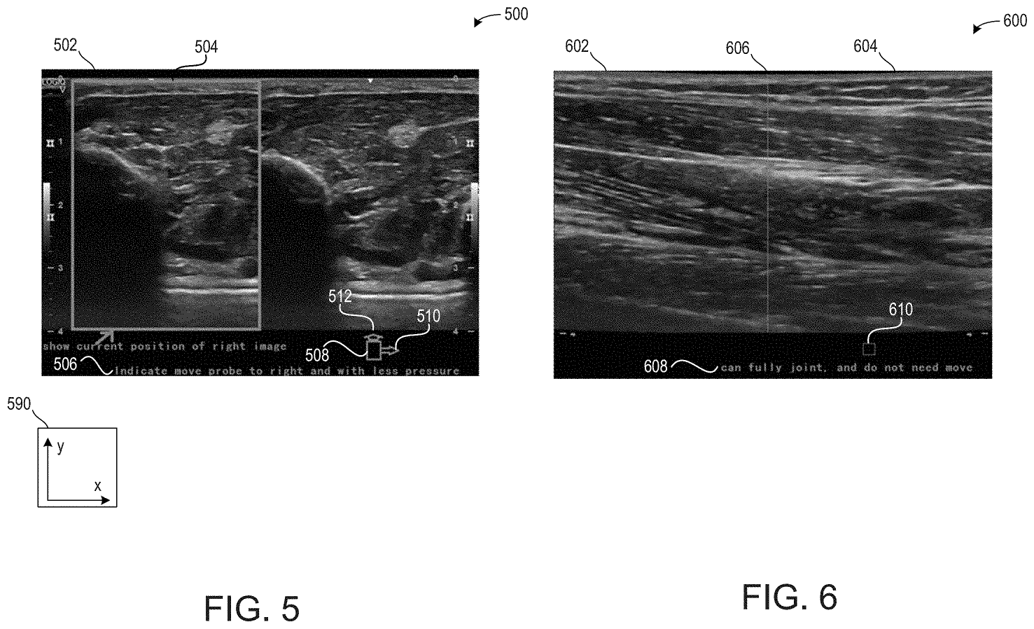

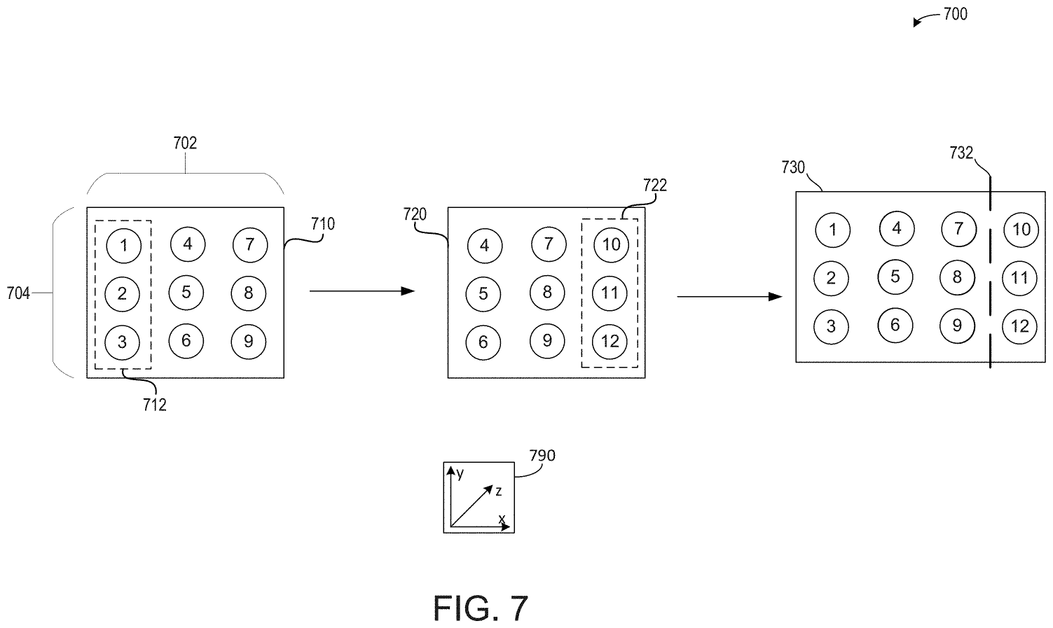

Methods for joining the figures are shown in FIGS. 3 and 4. FIG. 5 illustrates an embodiment of an image acquisition wherein a matching value between a first image and a second image of different sets of volume data is not greater than a threshold matching value. The user is instructed to move or continue moving the probe selected amounts in each of the horizontal and vertical planes to decrease an offset between the images, thereby increasing a matching value. FIG. 7 illustrates a further example of instructions provided to the user on a display device. FIG. 6 illustrates an embodiment of an image acquisition wherein the matching value of the first and second images is greater than the threshold matching value. As such, the images in the embodiment of FIG. 6 are joined along respective edges without overlapping.

FIG. 1 illustrates an example ultrasound system 100 that is configured for performing ultrasounds. In one example, the ultrasound system 100 is a hand-held ultrasound system. The ultrasound system includes a probe 101 that includes a housing 102 in which an ultrasound transducer array 104 is positioned. The transducer array is positioned at the scan head and is inserted into the tissue to be scanned, thus producing a sharper image relative to externally-applied probes because of the proximity of the transducer array to the tissue lining. The probe 101 may comprise a position tracking device including one or more position sensors (shown in FIG. 2) to allow position and orientation sensing for the transducer. Suitable position sensors (e.g., gyroscopic, magnetic, optical, radio frequency (RF)) may be used.

A fully-functional ultrasound engine for driving an ultrasound transducer and generating volumetric fetal, cardiac, trans-rectal, and intra-vascular ultrasound data from the scans in conjunction with the associated position and orientation information may be coupled to the probe. In some examples, the volumetric data may be gathered for obstetrics, cardiology, surgical guidance, vascular imaging, regional and anesthesia. For example the ultrasound engine may include a processor and memory and/or be implemented with a processor and memory. The ultrasound engine may be included as part of a scanning processor 106 coupled to the probe. The volumetric scan data can be transferred to another computer system for further processing using any of a variety of data transfer methods known in the art. A general purpose computer, which can be implemented on the same computer as the ultrasound engine, is also provided for general user interfacing and system control. The ultrasound system may be a self-contained stand-alone unit that may be moved from room to room, such as a cart-based system, a hand-carried system or other portable system. Further the ultrasound system of FIG. 1 may be remotely controlled, configured, and/or monitored by a remote station connected across a network.

FIG. 2 is a block diagram 200 schematically illustrating various system components of an ultrasound imaging system, including the probe 101, the scanning processor 106, a client device 230, and a remote device 240. In some embodiments, the scanning processor may be further coupled to a remote department information system, hospital information system, and/or to an internal or external network to allow operators at different locations to supply commands and parameters and/or gain access to image data (as explained in more detail below, remote device 240 is one non-limiting example of one such remote system to which the scanning processor may be coupled).

Referring first to the probe 101, the probe 101 comprises the transducer array 104. As explained above with respect to FIG. 1, the transducer array may be positioned within the housing, and the housing and transducer array may be configured to be moved manually by an operator during an ultrasound exam. The transducer array may include an array of transducer elements, such as piezoelectric elements, that convert electrical energy into ultrasound waves and then detect the reflected ultrasound waves.

The probe 101 may further include a memory 224. Memory 224 may be a non-transitory memory configured to store various parameters of the transducer array 104, such as transducer position information obtained from a position tracking device 228 of the probe, transducer usage data (e.g., number of scans performed, total amount of time spent scanning, etc.), as well as specification data of the transducer (e.g., number of transducer array elements, array geometry, etc.) and/or identifying information of the probe 101, such as a serial number of the probe. Memory 224 may include removable and/or permanent devices, and may include optical memory, semiconductor memory, and/or magnetic memory, among others. Memory 224 may include volatile, nonvolatile, dynamic, static, read/write, read-only, random-access, sequential-access, and/or additional memory. In an example, memory 224 may include RAM. Additionally or alternatively, memory 224 may include EEPROM.

Memory 224 may store non-transitory instructions executable by a controller or processor, such as controller 226, to carry out one or more methods or routines as described herein below. Controller 226 may receive output from various sensors of the tracking device 228 and trigger actuation of one or more actuators and/or communicate with one or more components in response to the sensor output. In one example, the tracking device 228 may include one or more position sensors, accelerometers, gyroscopes, pressure sensors, strain gauge sensors, and/or temperature sensors. The tracking device 228 may further include a reference marker detector configured to detect a reference marker positioned on a patient undergoing ultrasound scanning. The reference marker detector may be configured to detect a magnetic reference marker, optical reference marker, or other suitable reference marker positioned internally or externally of the patient. Prior to and/or during image acquisition, the position of the probe (in six degrees of freedom, including translation across and rotation about three perpendicular axes) relative to the reference marker may be determined from the output of the position sensor(s) of the tracking device 228 and stored in memory 224 and/or sent to the scanning processor 106. The output from the sensors may be used to provide feedback to an operator of the probe 101 (via a displayed user interface of scanning processor 106, client device 230, and/or another device). For example, the operator may be instructed to reposition the probe prior to image acquisition, if the probe is not located at a predetermined position. In another example or in addition, the operator may be instructed to adjust an angle, depth, and/or location of probe during scanning, such as described further herein.

Probe 101 may be in communication with scanning processor 106 to send raw scanned data to an image processor 212, for example. Additionally, data stored in memory 224 and/or output from tracking device 228 may be sent to scanning processor 106 in some examples. Further, various actions of the probe 101 (e.g., activation of the transducer elements) may be initiated in response to signals from the scanning processor 106. Probe 101 may optionally communicate with a display of the scanning processor and/or display 236 of the client device, in order to notify a user to reposition the probe 101, as explained above, or to receive information from a user, for example.

Turning now to scanning processor 106, it includes an image processor 212, storage 214, display output 216 configured to send information for display on a display device of the scanning processor 106 and/or another display device (such as a display of remote device 240), and ultrasound engine 218. Ultrasound engine 218 may drive activation of the transducer elements of the transducer array 104. Further, ultrasound engine 218 may receive raw image data (e.g., ultrasound echoes) from the probe 101. The raw image data may be sent to image processor 212 and/or to a remote processor (via a network, for example) and processed to form a displayable image of the tissue sample which may be sent to a coupled display device via the display output 216. It is to be understood that the image processor 212 may be included with the ultrasound engine 218 in some embodiments.

Information may be communicated from the ultrasound engine 218 and/or image processor 212 to a user of the ultrasound probe system via the display output 216 of the scanning processor 106. In one example, the user may include an ultrasound technician, nurse, or physician such as a radiologist. For example, after acquiring imaging data (e.g. ultrasound imaging data) in real time, an image may be generated from the data and displayed on a display device (of the scanning processor 106 or an operatively coupled device) via the display output 216. In another example, information relating to parameters of the scan, such as the progress of the scan, may be sent to the display via the display output 216. In another example, the display output 216 may output a user interface configured to display images or other information to a user. Further, the user interface may be configured to receive input from a user and send the input to the scanning processor 106. User input may be via a touch screen of a display in one example. However, other types of user input mechanisms are possible, such as a mouse, keyboard, etc.

Scanning processor 106 may further include storage 214. Similar to memory 224 of the probe, storage 214 may include removable and/or permanent devices, and may include optical memory, semiconductor memory, and/or magnetic memory, among others. Storage 214 may include volatile, nonvolatile, dynamic, static, read/write, read-only, random-access, sequential-access, and/or additional memory. Storage 214 may store non-transitory instructions executable by a controller or processor, such as ultrasound engine 218 or image processor 212, to carry out one or more methods or routines as described herein below. Storage 214 may store position information of the ultrasound probe as communicated via the tracking device 228. Storage 214 may store raw image data received from the ultrasound probe, processed image data received from image processor 212 or a remote processor, and/or additional information.

The scanning processor 106 and/or probe 101 may be communicatively coupled to a client device 230. The client device 230 may include a suitable computing device, such as a laptop, tablet, or mobile device. The client device 230 may include a controller 232, memory 234, display 236, and camera 238. The controller 232 may include an image processor and ultrasound engine, similar to the image processor and ultrasound engine of the scanning processor 106. In this way, the client device 230 may drive actuation of the elements of the transducer array 104 of the probe 101, receive raw image data from the probe 101, and/or process the raw image data into one or more images. The controller 232 may further execute instructions stored in memory 234 in order to carry out one or more of the methods described herein.

For example, the instructions may enable the controller to gather volume data in the form of a plurality of image slices. Each slice may correspond to a dimension and a time at which ultrasound data is acquired. References points of individual slices may be analyzed such that instructions via a user interface may be displayed to a user, wherein the instructions may include how and where to arrange the probe to gather a subsequent image slice. The image slices may be stitched together upon a dimension of the subsequent image sufficiently matching, via measurement and comparison of a matching value relative to a threshold matching value, the dimension of a previous image slice. The combination of the two image slices may produce a larger, 3D image of a target anatomical location.

Memory 234 may include volatile, nonvolatile, dynamic, static, read/write, read-only, random-access, sequential-access, and/or additional memory. Memory 234 may store non-transitory instructions executable by a controller or processor, such as controller 232, to carry out one or more methods or routines as described herein below. Memory 234 may store position information of the ultrasound probe as communicated via the tracking device 228 of the probe. Memory 234 may store raw image data received from the ultrasound probe, processed image data received from the image processor, and/or additional information.

The display 236 may display suitable information, such as one or more user interfaces, images, etc. The display 236 may display content sent from controller 232 or other suitable processor. The camera 238 may include both forward-facing and rear-facing image sensors each configured to acquire visible light and/or light-based depth images. Visible light images and/or light-based depth images may be displayed on display 236 in the form of still images and/or videos. In some examples, data from the forward- and/or rear-facing image sensors may also be used to determine direction/location and orientation data (e.g. from imaging environmental features) that enables position/motion tracking of the client device 230 in the real-world environment.

Client device 230 may further include a tracking device 239. Tracking device 239 may be configured to provide position and/or orientation data of the client device 230 to the controller 232. In one example, the tracking device may be configured as an inertial movement unit (IMU) including a three-axis or three-degree of freedom (3DOF) position sensor system. This example position sensor system may, for example, include three gyroscopes to indicate or measure a change in orientation of the client device within 3D space about three orthogonal axes (e.g., roll, pitch, and yaw). The orientation derived from the sensor signals of the IMU may be used to display, via the display 236, one or more augmented reality images with a realistic and stable position and orientation.

In another example, the IMU may be configured as a six-axis or six-degree of freedom (6DOF) position sensor system. Such a configuration may include three accelerometers and three gyroscopes to indicate or measure a change in location of the client device along three orthogonal spatial axes (e.g., x, y, and z) and a change in device orientation about three orthogonal rotation axes (e.g., yaw, pitch, and roll). In some embodiments, position and orientation data from the image sensors and the IMU may be used in conjunction to determine a position and orientation of the client device.

Client device 230 may be communicatively coupled to a remote device 240. Remote device 240 may include a suitable computing device usable to store and/or display acquired ultrasound images, and may also be communicatively coupled to scanning processor 106. Remote device 240 may be part of a picture archiving and communication system (PACS) that is configured to store patient medical histories, imaging data, test results, diagnosis information, management information, and/or scheduling information, for example. The remote device 240 may comprise a PACS server that includes computer-readable storage media suitable for storing image data for later retrieval and viewing at a PACS workstation, for example. Remote device 240 may include a controller 242, memory 244, and/or display 246. Memory 244 may include volatile, nonvolatile, dynamic, static, read/write, read-only, random-access, sequential-access, and/or additional memory. Memory 244 may store non-transitory instructions executable by a controller or processor, such as controller 242, to carry out one or more methods or routines as described herein below. Memory 244 may store position information of the ultrasound probe as communicated via the tracking device 228 of the probe. Memory 244 may store raw image data received from the ultrasound probe, processed image data received from the image processor, and/or additional information.

Methods described herein may illustrate a method for assisting a user to automatically join multiple images. The method may include directing a user to move the ultrasound probe, wherein the directing may include a direction in which to move the probe and a pressure in which to apply to the probe. In this way, instructions are provided for actuating a probe in a three-dimensional space while scanning so that two or more images may be accurately joined.

A purpose of joining two or more images together is to acquire a wide image for a long scan area. For example, to get a whole image for a relatively large tumor in a patient's abdomen and thus to more accurately measure a size of the tumor.

Previous examples of joining medical diagnostic images together forced a user to identify regions of interest in a first portion of a long scan area image. However, this step of the process may be difficult as medical diagnostic images are grey with no clear boundaries between scanned objects to a user, especially an untrained user. Thus, to mark regions of interest may be time consuming and/or inaccurate. Ultrasound data acquisition for a target area may occur from a plurality of positions. For example, images may be acquired from an anterior, a posterior, and/or a medial position, producing a large volume of data. It may be difficult for the user to combine each of these images in the large volume of data to produce a single, large image (e.g. the long scan area image).

Furthermore, ultrasound images may be dependent on a probe position, orientation, and pressure. Thus, moving the ultrasound probe along a patient's body may adjust an ultrasound image, holding the probe at various angles may adjust the ultrasound image, and a pressure applied to the patient's body with the ultrasound prove may adjust the ultrasound image, further complicating combining images acquired from different locations. By adjusting the ultrasound image, a plane, a location, and/or an angle at which a reference point of an anatomical position is visualize may change an appearance of the reference point. These three factors may increase a difficulty of aligning a second portion of the long scan area image with the first portion.

To solve the above described issues, the ultrasound diagnostic device may assist a user in joining two or more images. Instructions may be provided to a user as they acquire volume data so that images from the sets of volume data can be joined more accurately. Adjacent portions of the images may be continuously compared to calculate a matching value during scanning. The matching value may be compared to a threshold value. Images from the multiple sets of volume data may be joined if the matching value is greater than the threshold value. If the matching value is not greater than the threshold value, then instructions may be provided to recapture at least one of the adjacent portions of the images to increase the matching value. The instructions to the use may include one or more directions to move, an amount to move (in each of the one or more directions), one or more angles to move, one or more axes about which to rotate, and/or an amount of pressure to apply during scanning. Reference points between images acquired at different locations may be more accurately matched and the images stitched.

Turning now to FIG. 3, it shows a high-level flow chart illustrating a method 300 for joining disparate medical diagnostic images. Instructions for carrying out method 300 and the rest of the methods included herein may be executed by a controller based on instructions stored on a memory of the controller and in conjunction with signals received from sensors of the engine system, such as the sensors described above with reference to FIG. 2. The controller may employ engine actuators of the engine system to adjust engine operation, according to the methods described below.

The method 300 begins at 302, which includes acquiring a first image. The first image may be one of a plurality of images of a target region of a patient. The first image may be acquired by an ultrasound probe, such as ultrasound probe 101 of FIG. 1.

The method 300 proceeds to 304, which includes marking two or more reference points in the first image. The reference points may correspond to organs, bones, muscle insertions, and the like. As such, the reference points may correspond to known components of the patient anatomy. For example, a reference point may include one or more of a tendon, skeleton, a joint, an organ, a nerve, a blood vessel, a fetus, and the like. The reference points may be labeled on the image and/or may be stored in memory of a controller of the ultrasound device. As will be described in further detail below with respect to FIG. 4, the first image may be compared to a previously acquired image of the target region, wherein the reference points may be compared to previously evaluated reference points to determine the legitimacy and quality of the first image.

The method 300 proceeds to 306, which includes displaying a real-time probe location. The probe location may be displayed in a first plane and a second plane. The first plane, which may also be referred to as a horizontal plane, may illustrate a direction of movement along the patient's body. The second plane, which may also be referred to as a vertical plane, may illustrate a pressure of the probe against the patient's body and may be perpendicular to the first plane. In this way, the user may be aware of both a direction and a pressure of the probe on the patient's body so that a similar direction and a similar pressure may be applied during the acquisition of a subsequent image desired to join with the first image.

In one example, if the medical imaging is of a patient's abdomen, then the first plane, which includes the direction of movement, may be parallel to a coronal plane. The second plane may be perpendicular to the coronal plane and parallel to the sagittal or transverse planes. It will be appreciated that orientation of the first plane and the second plane may be adjusted based on a location of the medical imaging relative to the patient's body such that the first plane may be parallel to one of the coronal plane, sagittal plane, or the transverse plane.

The method 300 proceeds to 308, which includes acquiring a second image. In one example, the second image is an image of the target region directly adjacent to at least portions of the first image. In one example, the first image may be a first half of a final image and the second image may be a second half of the final image. Additionally or alternatively, the first image and the second image may be separate images to be joined together in the final image, wherein the first image and the second image share a common border. In one example, the first image may visualize a portion of a tumor or a portion of a reference point and the second image may visualize a remaining portion of the tumor or a remaining portion of the reference point.

The method 300 proceeds to 310, which includes displaying a real-time matching value. The matching value may correspond to an accuracy of a joining of the first image and the second image. That is to say, as a difference between the first image and the second image increases, the matching value decreases. In this way, a higher matching value may represent a smaller difference in horizontal and vertical directions in which the first and second images were acquired. The higher matching value may further correspond to a more accurate joining of the first and second images such that the portion of the patient's anatomy scanned may be more reliably measured and/or analyzed.

The method 300 proceeds to 312, which includes determining if the matching value is greater than a threshold value. The threshold value may be based on a plurality of previously joined images. In one example, the threshold value is a fixed value. For example, the threshold value may be equal to a score of 9 out of a score system with a highest value of 10 and a lowest value of 1. As another example, the threshold value may be a percentage, such as 85%. Additionally or alternatively, the threshold value may be a dynamic value, wherein the plurality of previously joined images continuously updates the threshold value as different combinations and/or joining of images are learned. Such an example is described in greater detail below.

If the matching value is not greater than the threshold value, then the method 300 proceeds to 314, which includes rescanning portions of the second image. Rescanning may include adjusting a force applied to second image area during the second image reacquisition. By adjusting the force, planes in which the first and second images are acquired may more closely match, thereby increasing the matching value.

If the matching value is greater than the threshold value, then the method 300 may proceed to 316 to join the first and second images. Joining the first and second images may include stitching the first and second images at a common border, wherein the common border is a central axis of a final image comprising the first and second images. That is to say, the region at which the first and second images are combined is a middle of the final image. The borders of the first and second images may be adjacent to one another along the horizontal axis. Additionally or alternatively, a reference point may extend across the first and second images such that the borders divide the reference point across the first and second images. In this way, the correct borders of the first and second images for joining may be selected.

Herein, the description more specifically describes the acquisition and joining of the first and second images based on one or more of an algorithmic method or a deep learning network. Broadly, a spatial transformation matric of two adjacent images is calculated in real-time. Some values in the matrix illustrate a translation, so an accumulation of the values may provide a moved distance in the horizontal and vertical directions and/or planes. As described above, the horizontal direction and/or plane does not include pressure and is generally parallel to a patient's skin (e.g., scanning surface of the patient). In one example, the patient's skin may be an interface surface and/or an acquisition surface and/or the scanning surface, wherein the ultrasound probe contacts the interface surface and movement along the interface surface may be described herein as movement in the horizontal direction. The vertical direction and/or plane includes pressure and is perpendicular to the scanning surface of the patient. A matching value between the first image and a second, current image is provided via one or more of the algorithmic method and the deep learning network.

During the scanning (e.g., image acquisition), images may be obtained via periodic sampling. If the sampling period is equal to some duration of time (T), then a current image may be F(mT), m∈R⁺, and a first, previously acquired image may be F(0). The feature point pairs [(xᵢ, yᵢ), (x′ᵢ, y′ᵢ)], i∈R between F(mT) and F((m−1)T) are obtained by scale invariant feature transformation (SIFT).

Noise rejection may be based on a robust least-square algorithm which may include directly calculating a spatial transformation matrix from the feature point pairs. A matrix, illustrated in formula 1 below, may be calculated if each of the feature point pairs are calculated without error.

$$[ {x}_{i} , {y}_{i} ] [ \begin{matrix}p a & p b \\ p c & p d\end{matrix} ] + [ pe , pf ] = [ {x}_{i}^{′} , {y}_{i}^{′} ] \qquad ( Formula 1 )$$

sₓ and s_{y} are the scaling factor in x and y axes, θ is the rotation angle between two images, pe,pf are the moving distance on x and y axes. In one example, the horizontal direction is parallel to the x-axis and the vertical direction is parallel to the y-axis. Formula 1 may be rewritten in a simplified, homogeneous form in the example of formula 2 below.

$$[ {x}_{i} , {y}_{i} , 1 ] [ \begin{matrix}p a & p b & 0 \\ p c & p d & 0 \\ p e & p f & 1\end{matrix} ] = [ {x}_{i}^{′} , {y}_{i}^{′} , 1 ] \qquad ( Formula 2 )$$

The resulting matrices and/or vectors may be represented by symbols, as shown below.

$$\begin{matrix}A = [ \begin{matrix}{x}_{1} & {y}_{1} & 1 \\ {x}_{2} & {y}_{2} & 1 \\ ⋮ & ⋮ & ⋮ \\ {x}_{n} & {y}_{n} & 1\end{matrix} ] & ( Matrix 1 ) \\ {A}^{′} = [ \begin{matrix}{x}_{1}^{′} & {y}_{1}^{′} & 1 \\ {x}_{2}^{′} & {y}_{2}^{′} & 1 \\ ⋮ & ⋮ & ⋮ \\ {x}_{n}^{′} & {y}_{n}^{′} & 1\end{matrix} ] & ( Matrix 2 ) \\ X = [ \begin{matrix}p a & p b & 0 \\ p c & p d & 0 \\ p e & p f & 1\end{matrix} ] & ( Matrix 3 )\end{matrix}$$

An error may exist in the feature point pairs, and in some examples, one or more of the feature point pairs may be erroneous. To prevent formula 1 from representing all feature point pairs, a matrix may be generated to decrease total error to a lowest possible value. Such a matrix may be represented by matrix 4 below.

$$E = {A}_{1} X - {A}_{2} = [ \begin{matrix}{e}_{1} \\ {e}_{2} \\ ⋮ \\ {e}_{n}\end{matrix} ] \qquad ( Matrix 4 )$$

Matrix 4 is calculated and pairs that are unable to be mapped by the matrix 4 are rejected. The rejection may be based on a Huber function, which may decrease an impact and/or magnitude of the wrong pairs. By doing this, feature point pairs that are wrong or that comprise errors may be rejected to increase an accuracy of the spatial transformation matrix. The Huber cost function is represented by formula 3, in combination with formula 4, below.

$$\begin{matrix}{arg min}_{X} cost = {\sum }_{i = 1}^{n} {∅}_{h u b} ( {e}_{i} ) & ( Formula 3 ) \\ {∅}_{hub} ( e ) = \{ \begin{matrix}{e}^{2} & \vert e \vert \leq M \\ M ( 2 \vert e \vert - M ) & \vert e \vert > M\end{matrix} & ( Formula 4 )\end{matrix}$$

Formula 3 may be a strictly convex function, wherein a Newton's method is used to solve matrix 4. A range of M and M is illustrated by plot 1 below.

Points pairs are incorrect based on a determination of formula 5 below.

∥[xᵢ,yᵢ,1]X−[x′ᵢ,y′ᵢ,1]∥₂>ξ (Formula 5);

wherein ξ is a hyperparameter.

The rejected erroneous feature point pairs may provide cleaned feature point pairs B₁, B₂, and may be used to resolve spatial transformation matrix X₂ with B₁, B₂ as part of a solution of the spatial transformation matrix based on a L₂−Norm approximation. By omitting the erroneous pairs, X₂ can have a analytical solution if a L₂−Norm approximation. The problem to solve is illustrated by formula 6 below

argminₓ_{₂} cost=∥B₂X₂−B₂∥₂ (Formula 6)

cost=(B₁X₂−B₂)^{T}(B₁X₂−B₂)=X₂^{T}B₁^{T}B₁X₂−B₂^{T}B₁X₂−X₂^{T}B₁^{T}B₂+B₂^{T}B₂ (Formula 7)

Formula 7 illustrates an expanded form of formula 6, wherein the cost function is a strictly convex function, and takes a minimum value at derivative equal to 0, as shown below in derivation 1.

$$\frac{\partial cost}{\partial {X}_{2}} = 2 {B}_{1}^{T} {B}_{1} {X}_{2} - 2 {B}_{1}^{T} {B}_{2} = 0 \\ {B}_{1}^{T} {B}_{1} {X}_{2} = {B}_{1}^{T} {B}_{2} \\ {X}_{2} = {( {B}_{1}^{T} {B}_{1} )}^{- 1} {B}_{1}^{T} {B}_{2} \qquad ( Derivation 1 )$$

In one example, if the surface of the patient is considered in a first plane (e.g., an x-plane and/or a horizontal plane), then the probe may be actuated along the x-plane from a first location to a second, different location. The value pe is the offset of the probe during a first period (e.g., a displacement of the probe from the first location to the second location). Therefore, the offset (e.g., a mismatch) between a first image and a current image may be obtained by accumulating pe of every period based on formula 8 below.

Lₜ=Σₘ₌₁^{t}peₘ (Formula 8)

The probe may be moved in a second plane (e.g., a y-plane and/or a vertical plane), wherein the probe is pressed or pulled along the y-plane, at a fixed location, in a direction perpendicular to the x-plane. Additionally or alternatively, the orientation of the probe may result in a pressing or pulling of the probe in a direction angled to the x-plane. At any rate, movement along the y-plane may result in a pressure change applied to the probe parallel to the y-plane. Any movement of the probe not parallel to the y-plane may be measured along the x-plane.

A change in pressure applied to the probe may result in a change of a scanned image. A scanned image with a range of depth responds to a pressure change as desired. In this way, the solution of a probe pressure change in one period may be similar to a probe offset. However, only point pairs within the depth range participate in the calculation, wherein the depth range may correspond to an ultrasound setting. In the example of pressure being monitored along the y-plane, an image offset between the first image and a current image may be obtained based on formula 9 below.

Fₜ=Σₘ₌₁^{t}pe′ₘ (Formula 9)

Following determination of the offset in the x-plane via formula 8 and the offset in the y-plane via formula 9, a matching value may be determined based on a combination of the offsets in the x- and y-planes. In one example, the matching value is a measurement of an accuracy of combining the first and second images. In one example, the accuracy is inversely proportional to a difference between a pixel intensity of the two images at a location at which the two images will be joined. As such, a relatively low difference between the pixel intensities (e.g., higher similarity) may indicate a relatively high matching degree. The matching value may be calculated via formula 11 in combination with formula 10 below.

$$d = \frac{1}{n} {\sum }_{i = 0}^{n} \vert {f}_{i}^{1} - {f}_{i}^{2} \vert \qquad ( Formula 10 )$$

$$Matching value = exp ( \frac{d}{D} ) ; \qquad ( Formula 11 )$$

wherein D is a hyperparameter.

As described above with respect to method 300, if the matching value is greater than a threshold matching value, then the two images may be joined. If the matching value is not greater than the threshold matching value, then the two images may not be joined and the operator may be asked to adjust a movement and/or pressure of the probe to decrease the offset in the x- or y-planes.

The image joining may also be executed via a deep learning network structure, such as a deep learning neural network. The deep learning network structure may be used in combination with or separately from the algorithmic method previously described. The deep learning network structure may comprise a plurality of previously acquired image pairs, wherein each pair comprise at least two ultrasound images at two different but near body parts. Near body parts may be defined as body parts where ultrasound images thereof include image pairs that share at least one common border.

The two images may be resized to one common image size (e.g., 448 height×448 width) and are then processed through a convolutional layer to generate a feature map and/or an activation map. The convolutional layer may be a VGG, GoogLeNet, ResNet, and the like as known to those of ordinary skill in the art. As one example, if the convolutional layer is GoogLeNet, then the size of the feature map is 14×14×1024. The two feature maps are combined together with a final size of 12×14×2048. A 14×14×2048×2 convolutional kernel is operated on the combined two feature maps without padding and the two values obtained are equal to an offet between the two images in horizontal and vertical direction parallel to the x- and y-planes, respectively. A portion of each feature map is cut from each feature map's edge, wherein the portion is 1×14×1024. The two portions are combined to a size of 1×14×2048, wherein a 1×14×2048×2 convolutional kernel layer is operated on the two combined portion without padding to get a two value activated softmax function which is substantially equal to a matching value.

For the offset output, smooth-L1 may be selected as the loss function represent by formula 11 below.

$$\begin{matrix}{smooth}_{l 1} = \{ \begin{matrix}0.5 {a}^{2} & \vert a \vert < 1 \\ \vert a \vert - 0.5 & \vert a \vert \geq 1\end{matrix} & ( Formula 11 ) \\ {Loss}_{offset} = {smooth}_{l 1} ( x - {x}_{t} ) + {smooth}_{l 1} ( y - {y}_{t} ) & ( Formula 12 )\end{matrix}$$

Formula 12 may determine a total offset value that includes the offsets in the x- and y-planes. The matching value may be calculated by selecting a cross-entropy as a loss function as shown by formula 13 below.

Loss_{match}=Σᵢ₌₁²−yₜⁱ log(yⁱ)−(1−yₜⁱ)log(1−yⁱ) (Formula 13)

A total loss may be equal to a weighted sum of formulas 12 and 13 shown below in formula 14.

Total loss=λLoss_{offset}+Loss_{match} (Formula 14)

The total loss may be compared to the threshold matching value as described above.

Turning now to FIG. 4, it shows a method 400 for determining reference points in two adjacent images, matching the reference points, and determining a matching value along a stitching edge of the adjacent images.

Method 400 begins at 402, which includes determining if an ultrasound image is being acquired. The ultrasound image is being acquired if the probe is moved along the horizontal or vertical direction against a patient. If the ultrasound image is not being acquired, then the method 400 proceeds to 404, which includes maintaining current operation and does not display an ultrasound image on a display device.

If an ultrasound image is being acquired, then the method 400 proceeds to 406, which includes displaying an ultrasound probe location in real-time on a screen of a display device. The probe may provide feedback regarding an image (e.g., a slice of a volume data) acquired along with a position of the probe relative to the patient for the display device to display the real-time image of the probe. For example, the display may illustrate an image currently being acquired via the probe along with a box or some other shape illustrating an exact position of the probe. Furthermore, the position of the probe may be illustrated with respect to the x- and y-planes. This may be illustrated via arrows or other shapes. In one example, a size of the box may increase in response to a pressure of the probe against the patient increasing.

The method 400 proceeds to 408, which includes detecting reference points in the acquired image. Detecting reference points may be conducted via the deep learning neural network and/or the algorithmic method described above. The reference points detected may correspond to organs, vessels, nerves, joints, and the like. Additionally or alternatively, if the patient comprises a tumor or other growth formerly identified during diagnostic imaging, the tumor or other form of growth (e.g., a fetus) may be learned and added to a reference point dataset of the patient.

The method 400 proceeds to 410, which comprise matching detected reference points between two adjacent images. Two adjacent images may be defined as two images that visualize a shared reference point from different locations, wherein the two images share a border or other shared featured. Additionally or alternatively, adjacent images may comprise a first image visualizes a first anatomical location and the second image visualizes a second anatomical location, and where the first and second anatomical locations are contiguous. For example, the probe may visualize a reference point from a primarily anterior location of the patient's body during a first image and from a primarily posterior location of the patient's body during a second image. Each of the first image and the second image may comprise portions taken from a lateral portion of the patient's body. As such, the first image and the second image may comprise one or more reference points that may match at the lateral portions of the images.

In one example, the first and second images correspond to slices of a volume of data. The images may be for a trans-rectal ultrasound procedure, wherein the first image is gathered from a lateral position at a first time and the second image is gathered from a posterior position at a second time, subsequent the first time.

The method 400 proceeds to 412, which comprises using a robust least square calculation to remove wrong matched reference points. This may include the Huber calculation, as described with respect to formulas 3, 4, and 5 above. This may increase an accuracy of formula 1 if the algorithmic calculation is used.

The method 400 proceeds to 414, which comprises calculating a spatial transformation matrix. The spatial transformation matrix may be substantially equal to the formula 1, wherein the accuracy of the spatial transformation matrix is increased by accounting for errors in the reference pairs.

The method 400 proceeds to 416, which include determining an offset in horizontal and vertical planes with respect to the adjacent images. The offset in the horizontal plane may be calculated via formula 8 and the offset in the vertical plane may be calculated via formula 9.

The method 400 proceeds to 418, which includes calculating a matching value. The matching value may be based on a combination of the offsets in the horizontal and vertical planes. Additionally or alternatively, the matching value may be based on a difference in pixel intensity at a stitching edge between adjacent images, wherein the stitching edge corresponds to an edge or a border of the first and second images that will merge.

Once the matching value is determined, the matching value may be compared to a threshold matching value, similar to 312 of method 300. If the matching value is greater than the threshold matching value, then the two images may be joined together. As the user is scanning and the matching value exceeds the threshold matching value, the user may be instructed to briefly halt movement of the probe so that the images may be merged together at their respective stitching edges. The user may then be instructed to continue actuating the probe following the merger.

Turning to FIG. 5, it shows an embodiment 500 of a display 502 illustrating to a user a current probe position 504, written instructions 506, and visual instructions 508. The current probe position 504 may be based on feedback from a position sensor coupled to the probe, wherein the position sensor may provide feedback to a controller of an ultrasound device. Based on each of the position and the image provided by the probe, the written instructions 506 and the visual instructions 508 may instruct the user to adjust the probe position to increase a matching value between a first image and a second image. As shown, the visual instructions 508 comprise arrows, wherein a magnitude and/or a size of the arrow may indicate a desired command. A first arrow 512 is parallel to an x-axis of axis system 590, which is substantially similar to the x-plane and/or the horizontal plane described above. A second arrow 514 is parallel to a y-axis of axis system 590, which is substantially similar to the y-plane and/or vertical plane described above. The first arrow 512 is larger than the second arrow 514, thereby indicating that it is desired to move the probe more to along the horizontal plane and less in the vertical plane to increase the matching value.

Turning now to FIG. 7, it shows an embodiment 700 illustrating an example of an instruction to a user to displace a probe along a horizontal plane. As is known to those of ordinary skill in the art, the probe of an ultrasound device may scan images in three-dimensions, illustrated via axis system 790 comprising an x-axis, a y-axis, and a z-axis perpendicular to each of the x- and y-axes. A horizontal plane may be arranged along the x- and y-axes. A vertical plane may be arranged along the y- and z-axes.

A width of the probe is illustrated via bracket 702. A depth at which the probe soundwaves may penetrate and acquire images is shown via bracket 704. First image 710 represents an image previously acquired by a user. The ultrasound device may receive the image and identify reference points within the image. In the example of FIG. 7, the first image 710 comprises nine reference points.

Following acquisition of the first image 710, the user may select a long scan mode or a wide scan mode. In response, the ultrasound device may project the first image 710 on a first half of a display device, such as display 236 of client device 230 of FIG. 2. A second image 720, which is desired to be acquired following the first image 710, may be displayed on a second half of the display device. As such, the first image 710 and the second image 720 are displayed side-by-side on the display device for the user to readily visualize a final image that will include each of the first and second images 710, 720.

In the example of FIG. 7, the user is instructed to more the probe along the horizontal plane equal to one-third a width of the probe. As such, the second image 720 may no longer include the reference points 1, 2, and 3, as shown by dashed box 712. Due to the displacement of the probe in the horizontal direction, new reference points 10, 11, and 12 are gained, as shown by dashed box 722. In the example of FIG. 7, the user is not instructed to adjust a displacement of the probe along the vertical plane in order to maintain a substantially uniform depth between the first and second images 710, 720.

Following acquisition of the second image 720, a matching value may be calculated. If the matching value is greater than the threshold matching value, then the first image 710 may be joined to the second image 720. In the example of FIG. 7, stitching the first image 710 to the second image 720 may occur at edge of the images between reference points 7-9 and reference points 10-12.

An example edge at which the first image 710 and the second image 720 may be joined and/or stitched is shown via dashed line 732 on a third image 730. In this way, redundant information may be omitted. As such, the third image 730 comprises each of the first image 710 and the second image 720, wherein the images are joined side-by-side with one another without overlapping. The user may select to continue imaging the patient, wherein further images may be added to the third image 730 to further increase a width of the image.

Turning now to FIG. 6, it shows an embodiment 600 of a first image 602 and a second image 604 being merge along a common stitching edge 606. In one example, the stitching edge 606 corresponds to a central axis of the merged final image comprising the first and second images. As shown, the first and second images are stitched together side-by-side without overlapping. In this way, the images are contiguous with one another such that anatomical locations visualized by each of the images are also contiguous. In some examples, if portions of the first and/or second images are redundant such that an overlap may occur, the portions may be cropped (e.g., remove). Additionally or alternatively, the portions may be used as a further reference point to further match the first and second images.

The embodiment 600 further comprises written instructions 608 and visual instructions 610. As shown, the written instructions 608 instruct the user that the images may be joined and to halt movement of the probe. The visual instructions 610 no longer provide arrows indicating a direction in which it may be desired to move the probe. In response, the images may be automatically joined and/or stitched. Automatically joining and/or stitching the images may include where no further input is provided by the user. As such, the user may not indicate reference points or manually join the images.

In this way, a medical imaging device, such as an ultrasound device, is configured to merge two images of a reference point. In one example, the images are acquired from disparate vantage points relative to a patient's body. The technical effect of merging the two images of the reference point is to provide a single, combined image of the reference point for medical analysis with a high degree of accuracy to accurately diagnose a condition of the patient.

An embodiment of a method, comprises instructing a user to navigate an ultrasound probe via a movement in a horizontal plane or a pressure in a vertical plane based on a previously acquired first image at a first anatomical location while acquiring a second image at a second, different anatomical location and automatically joining the first and second images together.

A first example of the method further includes where the first and second images are generated from volume data, and wherein automatically joining the first and second images together is in response to a matching value of the images being greater than a threshold matching value.

A second example of the method, optionally including the first example, further includes where the matching value is based on an offset of the first and second images along the horizontal plane and the vertical plane, wherein the matching value increases as the offset decreases.

A third example of the method, optionally including one or more of the previous examples, further includes where the horizontal plane is parallel to a scanning surface of a patient.

A fourth example of the method, optionally including one or more of the previous examples, further includes where the vertical plane is perpendicular to a scanning surface of a patient.

A fifth example of the method, optionally including one or more of the previous examples, further includes where the first image is contiguous with the second image.

A sixth example of the method, optionally including one or more of the previous examples, further includes where the first and second anatomical locations are contiguous.

An embodiment of a system, comprises an ultrasound device comprising a probe and a display device and a controller with computer-readable instructions stored on non-transitory memory thereof that when executed enable the controller to display a first image on the display device, display a second image, different than the first image on the display device, identify shared reference points between the first image and the second image, calculate a matching value including along edges of the first image and the second image, and stitch the first image and the second image along respective edges in response to the matching value being greater than a threshold value.

A first example of the system further includes where the first image and the second image do not overlap.

A second example of the system, optionally including the first example, further includes where the instructions further enable the controller to display a location of the probe relative to the first image and the second image on the display device.

A third example of the system, optionally including one or more of the previous examples, further includes where the instructions further enable the controller to display instructions on the display device, wherein the instructions are configured to instruct a user to actuate the probe along a horizontal plane parallel to a surface of a patient's body and a vertical plane perpendicular to the surface of the patient's body to increase the matching value if the matching value is not greater than the threshold value.

A fourth example of the system, optionally including one or more of the previous examples, further includes where the instructions further enable the controller to identify reference points in the first image and the second image, wherein the reference points include one or more of a tendon, a skeleton, a joint, an organ, a nerve, a blood vessel, a tumor, and a fetus.

A fifth example of the system, optionally including one or more of the previous examples, further includes where the first image and the second image share at least one reference point.

An additional embodiment of a method, comprises displaying a first image acquired via an ultrasound probe on a display screen, displaying a location of the ultrasound probe relative to a coordinate system of the first image on the display screen, instructing a user a direction in a first plane in which to navigate the ultrasound probe while acquiring a second image, instructing the user a direction in a second plane, perpendicular to the first plane, in which to navigate the probe, calculating and displaying a matching value based on offsets in the first and second planes between the first image and the second image, and combining the first image and the second image side-by-side at edges of the first and second images.

A first example of the method further includes where instructing the user the direction to navigate the ultrasound probe in the first plane further comprises an arrow, further comprising increasing a size of the arrow to increase an instructed amount in which the ultrasound probe is desired to move, wherein the first plane is parallel to a scanning surface.

A second example of the method, optionally including the first example, further includes where instructing the user the direction to navigate the ultrasound probe in the second plane further comprises an arrow, the method further comprising decreasing a size of the arrow to decrease an instructed pressure which is desired to be applied to the ultrasound probe, wherein the second plane is perpendicular to the first plane, wherein an amount of size decrease is based on ultrasound volume data.

A third example of the method, optionally including one or more of the previous examples, further includes displaying the first and second images side-by-side on the display screen prior to combining the first and second images.

A fourth example of the method, optionally including one or more of the previous examples, further includes identifying reference points in each of the first image and the second image.

A fifth example of the method, optionally including one or more of the previous examples, further includes combining the first image and the second image is in response to a matching value being greater than a threshold matching value, wherein the matching value is calculated based on an offset between the first and second images in the first plane and the second plane.

A sixth example of the method, optionally including one or more of the previous examples, further includes where the matching value is further based on a difference of a pixel intensity at edges of the first and second images, wherein the matching value decreases as the difference increases.

As used herein, an element or step recited in the singular and proceeded with the word “a” or “an” should be understood as not excluding plural of said elements or steps, unless such exclusion is explicitly stated. Furthermore, references to “one embodiment” of the invention do not exclude the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The control methods and routines disclosed herein may be stored as executable instructions in non-transitory memory and may be carried out by the control system including the controller in combination with the various sensors, actuators, and other engine hardware. The specific routines described herein may represent one or more of any number of processing strategies such as event-driven, interrupt-driven, multi-tasking, multi-threading, and the like. As such, various actions, operations, and/or functions illustrated may be performed in the sequence illustrated, in parallel, or in some cases omitted. Likewise, the order of processing is not necessarily required to achieve the features and advantages of the example embodiments described herein, but is provided for ease of illustration and description. One or more of the illustrated actions, operations and/or functions may be repeatedly performed depending on the particular strategy being used. Further, the described actions, operations and/or functions may graphically represent code to be programmed into non-transitory memory of the computer readable storage medium in the engine control system, where the described actions are carried out by executing the instructions in a system including the various engine hardware components in combination with the electronic controller.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
instructing a user to navigate an ultrasound probe via a movement in a horizontal plane or a pressure in a vertical plane based on a previously acquired first image at a first anatomical location while acquiring a second image at a second, different anatomical location; and
automatically joining the first and second images together.

2. The method of claim 1, wherein the first and second images are generated from volume data, and wherein automatically joining the first and second images together is in response to a matching value of the images being greater than a threshold matching value.

3. The method of claim 2, wherein the matching value is based on an offset of the first and second images along the horizontal plane and the vertical plane, wherein the matching value increases as the offset decreases.

4. The method of claim 1, wherein the horizontal plane is parallel to a scanning surface of a patient.

5. The method of claim 1, wherein the vertical plane is perpendicular to a scanning surface of a patient.

6. The method of claim 1, wherein the first image is contiguous with the second image.

7. The method of claim 1, wherein the first and second anatomical locations are contiguous.

8. A system, comprising:
an ultrasound device comprising a probe and a display device; and
a controller with computer-readable instructions stored on non-transitory memory thereof that when executed enable the controller to:
display a first image on the display device;
display a second image, different than the first image on the display device;
identify shared reference points between the first image and the second image;
calculate a matching value including along edges of the first image and the second image; and
stitch the first image and the second image along respective edges in response to the matching value being greater than a threshold value.

9. The system of claim 8, wherein the first image and the second image do not overlap.

10. The system of claim 8, wherein the instructions further enable the controller to display a location of the probe relative to the first image and the second image on the display device.

11. The system of claim 8, wherein the instructions further enable the controller to display instructions on the display device, wherein the instructions are configured to instruct a user to actuate the probe along a horizontal plane parallel to a surface of a patient's body and a vertical plane perpendicular to the surface of the patient's body to increase the matching value if the matching value is not greater than the threshold value.

12. The system of claim 8, wherein the instructions further enable the controller to identify reference points in the first image and the second image, wherein the reference points include one or more of a tendon, a skeleton, a joint, an organ, a nerve, a blood vessel, a tumor, and a fetus.

13. The system of claim 12, wherein the first image and the second image share at least one reference point.

14. A method, comprising:
displaying a first image acquired via an ultrasound probe on a display screen;
displaying a location of the ultrasound probe relative to a coordinate system of the first image on the display screen;
instructing a user a direction in a first plane in which to navigate the ultrasound probe while acquiring a second image;
instructing the user a direction in a second plane, perpendicular to the first plane, in which to navigate the probe;
calculating and displaying a matching value based on offsets in the first and second planes between the first image and the second image; and
combining the first image and the second image side-by-side at edges of the first and second images.

15. The method of claim 14, wherein instructing the user the direction to navigate the ultrasound probe in the first plane further comprises an arrow, further comprising increasing a size of the arrow to increase an instructed amount in which the ultrasound probe is desired to move, wherein the first plane is parallel to a scanning surface.

16. The method of claim 14, wherein instructing the user the direction to navigate the ultrasound probe in the second plane further comprises an arrow, the method further comprising decreasing a size of the arrow to decrease an instructed pressure which is desired to be applied to the ultrasound probe, wherein the second plane is perpendicular to the first plane, wherein an amount of size decrease is based on ultrasound volume data.

17. The method of claim 14, further comprising displaying the first and second images side-by-side on the display screen prior to combining the first and second images.

18. The method of claim 14, further comprising identifying reference points in each of the first image and the second image.

19. The method of claim 14, wherein combining the first image and the second image is in response to a matching value being greater than a threshold matching value, wherein the matching value is calculated based on an offset between the first and second images in the first plane and the second plane.

20. The method of claim 19, wherein the matching value is further based on a difference of a pixel intensity at edges of the first and second images, wherein the matching value decreases as the difference increases.
